(19) United States
(12) Patent Application Publication
(10) Pub. No.: US 2006/0246117 A1
(43) Pub. Date: Nov. 2, 2006

(54) Animal husbandry method for administering a vitamin E derivative and formulation

(75) Inventors: Stefan Markus Jakob, Montlucon (FR); Pierre-Andre Geraert, Rochecorbon (FR)

Correspondence Address: OLIFF & BERRIDGE, PLC, P.O. BOX 19928, ALEXANDRIA, VA, 22320, US

(73) Assignee: ADISSEO FRANCE S.A.S., Antony (FR)

(21) Appl. No.: 11/408,985

(22) Filed: Apr. 24, 2006

Related U.S. Application Data
(63) Continuation-in-part of application No. PCT/FR04/02719, filed on Oct. 22, 2004.

(30) Foreign Application Priority Data
Oct. 22, 2003 (FR) 0312352

(51) Int. Cl. A23K 1/165; A61K 31/355

(52) U.S. Cl. 424/442; 514/458; 426/635

(57) ABSTRACT

The non-therapeutic animal husbandry method according to the invention consists in administering, by the oral route to a monogastric animal, a stable derivative of vitamin E, either on its own or mixed with an additive and/or a feedstuff, with the said derivative being hydrolyzable into the assimilable form of vitamin E, and being administered to the animal in the presence of at least one alimentary emulsifier which is selected from the non-ethoxylated esters of sorbitol and fatty acids. The invention also relates to a formulation which comprises a stable derivative of vitamin E and at least one said alimentary emulsifier and to the use of an alimentary emulsifier in animal nutrition.

The present invention relates generally to the administration of vitamin E to reared monogastric animals such as pigs and poultry, but also fish and prawns.

Vitamin E, or d-α-tocopherol, is mainly found in nature in vegetable oils; it is obtained at the end of different routes of synthesis in the racemic form d,l-α-tocopherol (abbreviated to Tol). In the native state, vitamin E is an oily lipophilic liquid which is miscible in any proportions in any hydrophobic or lipid phase. It is extremely unstable and easily oxidizable and loses the bulk of its biological activity when it is in the oxidized state. Its bioavailability in animals does not exceed 50% when it is administered by the oral route since, because it is rapidly oxidized, it is for the most part absorbed in an oxidized, inactive form. Consequently, when it is administered by the oral route, vitamin E is in the form of a more stable derivative, generally selected from the esters, for example the acetate, and the salts, of vitamin E.

Before the prior art which is closest to the invention, and the problems encountered by this prior art, are dealt with, and the invention is presented, a definition of bioavailability as it is understood in the remainder of the description is given as follows. The bioavailability of vitamin E or of a derivative of vitamin E is represented by the concentration of vitamin E which is released in the blood in relation to the concentration of vitamin E which is present in the animal's ration or in relation to the concentration, expressed in vitamin E equivalent, of the vitamin E derivative introduced into the animal's ration, when a derivative of the vitamin is administered. This representation of the bioavailability of vitamin E therefore takes into account the absorption of the vitamin E, or of the derivative of vitamin E, in the intestine during digestive passage.

According to T. Julianto et al., International Journal of Pharmaceutics, 200 (2000) pp 53-57, the authors prepared a solution of vitamin E in palm oil, with this solution being emulsified in a mixture of emulsifiers; after the solution had been diluted in water, the authors then examined the bioavailability, in humans, of the vitamin E in such a solution as compared with the bioavailability of vitamin E when supplied in the form of capsules. According to these authors, the bioavailability of vitamin E is three times greater when the vitamin E is supplied in the above-described solution. Considering the instability of vitamin E in non-esterified form, a solution of this nature cannot be used in a feed supplement.

A variety of feed supplements for animals, in particular marketed by the applicant, are known in which the vitamin E is administered in the form of vitamin E acetate, or d,l-α-tocopheryl acetate (Tac), on different supports, as, for example, while being adsorbed on silica, or in different physical forms, for example while being in the form of an oil-in-water emulsion.

Since the H. E. Gallo-Torres, Lipids (1970) vol. 5, No. 4, pp 379-384 publication, it is known that Tac is not assimilated directly in humans or animals but is in fact hydrolyzed to vitamin E in the gastrointestinal tract by the action of pancreatic enzymes termed cholesterol ester hydrolases (abbreviated to CEHs), with vitamin E finally being absorbed through the intestinal wall. However, Tac has only limited bioavailability, for example at best 40%, whatever the animal in question.

The object of the present invention is therefore to improve the bioavailability of vitamin E derivatives, in particular of Tac.

It was discovered, entirely surprisingly, that the presence of a particular emulsifier made it possible to significantly increase the bioavailability of a derivative of vitamin E, with the said derivative being able to be hydrolyzed into the assimilable form of vitamin E. This emulsifier is alimentary and is selected from the non-ethoxylated esters of sorbitol and fatty acids.

The Russian patent SU-1 676 572 discloses a composition which comprises vitamin E acetate, a polyethoxylated emulsifier and ethanol. It is added to poultry drinking water in a proportion which ensures a daily absorption of vitamin E of the order of 3 mg over given periods of time. The authors observe an assimilability of vitamin E, a rate of survival and an increase in weight of the poultry which are slightly greater than those obtained with conventional compositions.

The problem of bioavailability of vitamin E in reared animals, when administered in their rations, remains because the levels obtained with the known compositions are too low.

The applicant has demonstrated an effect of an emulsifier according to the invention on the vitamin E bioavailability of a composition according to the invention which is appreciably superior, particularly as compared with a polyethoxylated emulsifier. The applicant has furthermore discovered that the said emulsifier promotes the hydrolysis, in the gastro-intestinal tract, of the vitamin E derivative into its assimilable form, with this phenomenon having a favourable influence on the release of bioavailable vitamin E.

The invention is explained below in more detail; its advantages will then be illustrated in the examples, in particular comparative examples.

According to a preferred variant of the invention, the emulsifier is selected from long-chain fatty acid esters, for example those having a saturated or unsaturated hydrocarbon chain of at least 11 carbon atoms.

Thus, the invention firstly relates to an animal husbandry method which makes it possible to administer, to reared monogastric animals, a formulation of a vitamin E derivative in which, at one and the same time, the vitamin E is protected until it reaches its site of absorption and is bioavailable to a high degree. The method according to the invention is a non-therapeutic method which consists in administering, by the oral route to reared monogastric animals, a stable derivative of vitamin E either on its own or mixed with an additive and/or a feedstuff, with the said derivative being hydrolyzable into the assimilable form of vitamin E, in the presence of at least one alimentary emulsifier selected from the non-ethoxylated esters of sorbitol and fatty acids.

A fatty acid according to the invention is preferably understood as being a monocarboxylic acid which comprises a hydrocarbon chain having n carbon atoms, with n being an integer varying from 0 to 30 and the said chain being saturated or unsaturated. According to a more advantageous variant, the fatty acids according to the invention have a saturated or unsaturated hydrocarbon chain of at least 11 carbon atoms.

According to the method of the invention, the derivative of vitamin E derivative and the emulsifier can be administered to the animal concomitantly or sequentially, for example with the emulsifier being supplied to the animal before the vitamin E derivative. Thus, the vitamin E derivative and/or the emulsifier(s) can be mixed with the feedstuff.

The invention also relates to an alimentary formulation for animal nutrition which makes it possible, in particular, to implement the above method, which formulation comprises a stable derivative of vitamin E, with the said derivative being hydrolyzable into the assimilable form of vitamin E, and at least one alimentary emulsifier selected from the non-ethoxylated esters of sorbitol and fatty acids. According to one variant of the invention, it is preferable to use two alimentary emulsifiers which correspond to the above definition.

A formulation according to the invention preferably satisfies at least one of the following characteristics.

The ratio by weight of the emulsifier, or of the emulsifiers to the vitamin derivative can vary from 10/1 to 1/200; advantageously, it is between 1/5 and 1/100.

The vitamin E derivative is preferably a vitamin E ester, in particular vitamin E acetate.

A preferred emulsifier is a sorbitol ester selected from the monolaurate (SML), the monopalmitate (SMP), the monostearate (SMS), the monooleate (SMO) and the tristearate. The monooleate is preferred.

As the examples below will demonstrate, an advantageous formulation according to the invention comprises vitamin E acetate and at least one emulsifier selected from sorbitol monolaurate and sorbitol monooleate.

Another part of the subject-matter of the invention is the use of an alimentary emulsifier selected from the non-ethoxylated esters of sorbitol and fatty acids for preparing an alimentary formulation for animal nutrition which is based on a vitamin E derivative, with the said fatty acids advantageously corresponding to the aforementioned definition.

Yet another part of the subject-matter of the invention is the use of an above-described alimentary emulsifier for increasing the bioavailability of vitamin E in a reared monogastric animal, with the said derivative being hydrolyzable into the assimilable form of vitamin E.

The examples below illustrate the influence of an emulsifier according to the invention on the bioavailability of a derivative of vitamin E, its superior effect as compared with that of the known emulsifiers, and its influence on the hydrolysis of the vitamin E derivative, with the support of the drawings in which:

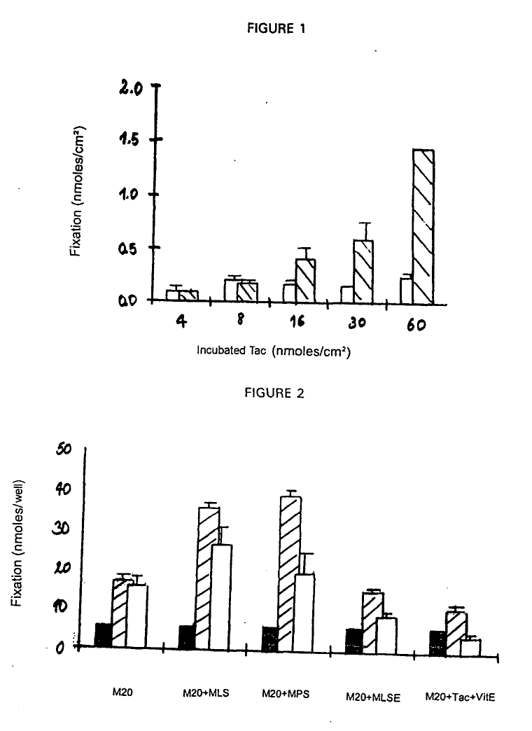

FIG. 1 is a graph depicting the quantity, in vitamin E equivalent, in nmoles/cm², which is fixed by a cell culture in dependence on the quantity, in nmoles/cm², of incubated Tac, with the said Tac being incubated without SMO (empty columns) and with 0.05% SMO (hatched columns).

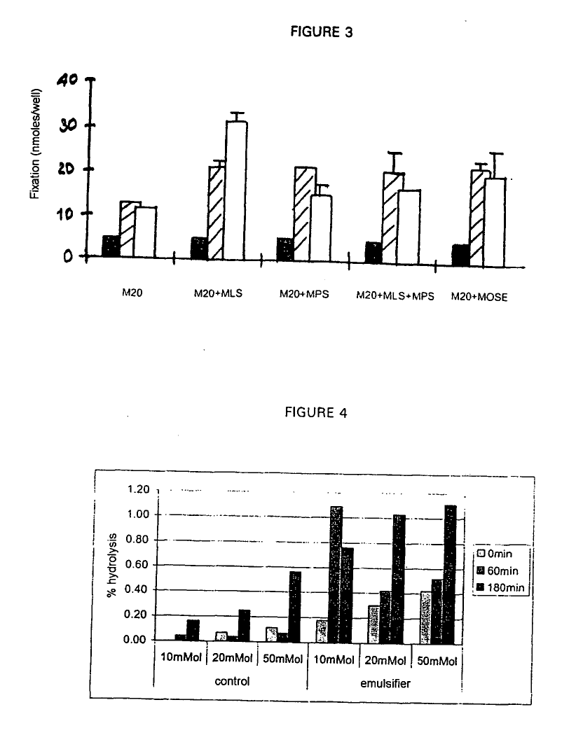

FIGS. 2 and 3 are graphs depicting the quantity fixed, in vitamin E equivalent, corresponding to retinyl acetate (Rac, an internal reference), in black columns, to Tac in hatched columns and to d,l-α-tocopherol in empty columns, in dependence on the absence or presence, and the nature, of the emulsifier which is incubated with the Tac. In accordance with FIG. 2, the emulsifier/vitamin E equivalent ratio is 6 while it is 1 in accordance with FIG. 3.

FIG. 4 depicts the degree of hydrolysis of Tac into Tol in dependence on the incubation time and under the conditions described in Example 5, 5.2).

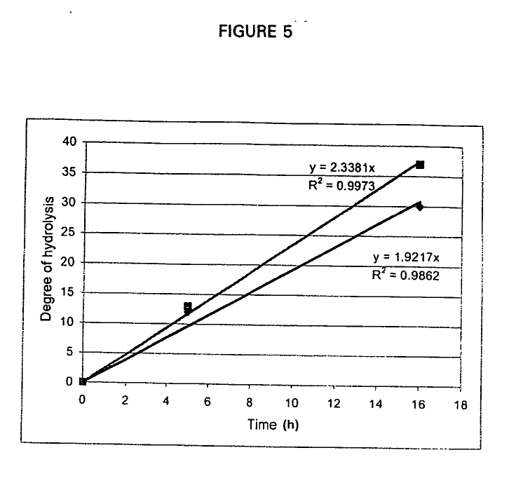

FIG. 5 depicts the degree of hydrolysis of Tac into Tol in dependence on the incubation time and under the conditions described in the Example 5, 5.1).

### EXAMPLE 1

### Influence of the Emulsifiers on the Absorption of Vitamin E Acetate (Tac) in an in vitro Cell System

By means of performing assays in vitro on an appropriate cell model, the quantity of Tac, expressed in vitamin E equivalent, which is fixed by the cell is measured by incubating a corresponding cell culture in the presence of Tac on its own, on the one hand, and, on the other hand, in the presence of Tac together with one or more alimentary emulsifiers according to the invention.

The cell model which is selected is a parental cell line which is designated CaCo-2 and which is available or accessible, under the reference HTB-37, from the American Type Culture Collection (ATCC).

In accordance with this assay, a cell culture of the previously identified cell line is incubated for 3 hours in the presence of micelles consisting of a suspension of Tac in water.

The quantity of micelles which is introduced into the cell culture is measured in nmoles of Tac/cm² and varies from 4 to 60 nmoles/cm².

For each concentration of Tac which has thus been introduced into the cell culture, the quantity of Tac which is fixed by the cell culture, and which is expressed in vitamin E equivalent in nmoles/cm², is on the one hand measured without emulsifier having been introduced into the said culture and on the other hand measured in the presence of a quantity of 0.05% (m/v) of the emulsifier SMO (sorbitan monooleate), with this percentage being obtained by dividing the mass of the emulsifier which has been introduced into the culture by the volume of micelles which have been introduced into the same culture.

The graph shown in FIG. 1 depicts the quantity of fixed Tac, expressed in vitamin E equivalent in nmoles/cm², as compared with the quantity of incubated Tac, expressed in nmoles/cm², with the empty columns relating to the micelles which were introduced without SMO and the hatched columns relating to the micelles which were introduced with 0.05% SMO. It can be seen that the quantity of corresponding vitamin E equivalent increases in proportion to the quantity of Tac which is incubated, up to the point of observing, in the final assay, a degree of fixation of the Tac by the cells which is six times greater when the emulsifier SMO is used.

### EXAMPLE 2

### Influence of the Emulsifiers on the Absorption of Vitamin E Acetate (Tac), and on the Absorption of Vitamin E, in an in vitro Cell System

2.1) Experimental Protocol:

Absorption assays are carried out, using the same cell line as that described above, by incubating the cells for 3 hours in a physiological medium which is similar to the intestinal medium and which contains cholesterol ester hydrolase (CEH), the pancreatic enzyme which hydrolyzes the Tac into vitamin E in the animal, and sodium taurocholate, which represents the bile salts. This medium comes in the form of an oil-in-water emulsion, i.e. in the form of micelles, and is commercially identified under the reference M20. The Tac, and, where appropriate, the emulsifier being tested, are placed in this medium. The concentration, in vitamin E equivalent, which is introduced into each well in the form of Tac is 156 μM.

Three six-well plates (representing, in the case of each well, an area of 9.6 cm²), containing the abovementioned medium together with respectively different emulsifiers, are used for each assay. Each experiment is therefore carried out three times.

2.2) Ratio by Weight of the Added Emulsifier to the Tac (in Vitamin E Equivalent) of the M20=Approximately 6:

In accordance with the present assay, the concentration of the emulsifier which is introduced into the M20 medium is 0.05%, expressed in mass of emulsifier(s) per volume of M20, with this corresponding approximately to a ratio by weight of the added emulsifier to the Tac of the M20 of approximately 6.

Six experiments are carried out, with each being repeated three times, using a protocol comprising three hours of incubation, as follows:
1) M20 on its own, at the rate of 2 mM of M20 per well; the Tac is therefore on its own without emulsifier;
2) M20+SML (sorbitan monolaurate) emulsifier
3) M20+SMP (sorbitan monopalmitate) emulsifier
4) M20+ESML (polyoxyethylated sorbitan monolaurate) emulsifier
5) M20+a mixture of 156 μM of Tac and 156 μM of d,l-α-tocopherol,
6) an internal reference termed Rac (retinyl acetate) for verifying the analysis.

For each assay, and expressed in nmoles of vitamin E equivalent fixed per well, the graph shown in FIG. 2 depicts:
the quantity of Rac, shown in black columns,
the quantity of Tac fixed by the cells, shown in the hatched columns, and
the quantity of vitamin E which has been obtained by hydrolysis and then fixed, shown in the empty columns.

As demonstrated by assays 2) and 3), the quantity of vitamin E absorbed by the cell culture is increased by approximately 50% as compared with that absorbed from M20 on its own.

By contrast, it is observed with assay 4), as compared with assay 1) without emulsifier, that the emulsifier ESML, corresponding to the ethoxylated emulsifier 2) (SML), has a tendency to prevent the absorption of the Tac and of the vitamin E.

Unexpectedly, it is found that ethoxylation of the emulsifier inhibits the favourable influence of an emulsifier according to the invention on the absorption of Tac and of vitamin E.

2.3) Ratio by Weight of the Added Emulsifier to the Tac (in Vitamin E Equivalent) of the M20=Approximately 1:

In accordance with the present assay, the concentration of the emulsifier which is introduced into the M20 medium is 0.01%, expressed in mass of emulsifier(s) as compared with the volume of M20, with this corresponding approximately to a ratio by weight of the added emulsifier to the Tac of the M20 of approximately 1.

Six experiments are carried out, with each being repeated three times, using a protocol which comprises three hours of incubation, as follows:
1) M20 on its own, at the rate of 2 mM of M20 per well; this therefore represents sodium taurocholate and Tac on its own
2) M20+SML (sorbitan monolaurate) emulsifier
3) M20+SMP (sorbitan monopalmitate) emulsifier
4) M20+SML emulsifier and SMP emulsifier
5) M20+SMO (sorbitan monooleate) emulsifier
6) the internal reference, i.e. Rac, for verifying the analysis.

For each assay, and expressed in nmoles of vitamin E equivalent fixed per well, the graph shown in FIG. 3 depicts:
the quantity of Rac, shown in black columns,
the quantity of Tac fixed by the cells, shown in the hatched columns, and
the quantity of vitamin E, obtained by hydrolysis and then fixed, shown in the empty columns.

Even at a lower ratio of emulsifier to the quantity of Tac (as compared with 2.2)), it can be seen that an emulsifier according to the invention has an influence on the absorption of Tac and of vitamin E.

### EXAMPLE 3

### Comparison of the Influence of the Emulsifiers According to the Invention and of the Corresponding Ethoxylated Emulsifiers on the Absorption of Tac and Vitamin E in an in vitro Cell System

3.1) Experimental Protocol:

The general experimental conditions of the protocol described in Example 2, 2.1) are identical apart from the fact that the area of the wells and the concentration of Tac introduced into the M20 medium differ. The area of the wells is 6.5 cm² and the concentration of Tac is 23.7 μM/cm².

3.2) Comparison of SML, SMP and ESML:

Four experiments are carried out, with each being repeated three times, using a protocol which comprises one hour of incubation at 37° C., as follows:
1) M20 on its own, at the rate of 2 mM; the Tac is therefore on its own without emulsifier;
2) M20+SML (sorbitan monolaurate) emulsifier
3) M20+SMP (sorbitan monopalmitate) emulsifier
4) M20+ESML (ethoxylated sorbitan monolaurate)

In the case of each of the assays 2), 3) and 4), the ratio by weight of the added emulsifier to the Tac (in vitamin E equivalent) is 1:6.9.

The results obtained are shown in Table 1 below:

TABLE 1

| | Absorption (nmol/cm²) | | |
|---|---|---|---|
| | Tol | Tac | Vitamin E |
| 1) Control | 1.77 | 1.68 | 3.45 |
| 2) Control + SML | 3.75 | 2.81 | 6.56 |
| 3) Control + SMP | 4.06 | 2.08 | 6.15 |
| 4) Control + ESML | 1.66 | 0.97 | 2.63 |

With the support of these results, it is noted that the emulsifiers according to the invention increase the absorption of Tol or of Tac, in vitamin E equivalent, by a factor of 1.9, in the case of SML, and by a factor of 1.78, in the case of SMP, as compared with the control assay 1) without emulsifier.

By contrast, it is observed that the ethoxylated emulsifier (MLSE) decreased the absorption of vitamin E by a factor of 0.76 as compared with assay 1).

3.3) Comparison of SML, SMP and MOSE:

Four experiments are carried out, with each being repeated three times, using a protocol which comprises one hour of incubation at 37° C., as follows:
1) M20 on its own; the Tac is therefore on its own without emulsifier;
2) M20+SML (sorbitan monolaurate) emulsifier
3) M20+SMP (sorbitan monopalmitate) emulsifier
4) M20+MOSE (ethoxylated sorbitan monooleate)

In the case of each of the assays 2), 3) and 4), the ratio by weight of the added emulsifier to the Tac (in vitamin E equivalent) is 1:6.9.

The results which were obtained are shown in Table 2 below:

TABLE 2

| | Absorption (nmol/cm²) | | |
|---|---|---|---|
| | Tol | Tac | Vitamin E |
| 1) Control | 1.29 | 2.31 | 3.60 |
| 2) Control + SML | 3.05 | 3.88 | 6.93 |
| 3) Control + SMP | 3.33 | 3.18 | 6.51 |
| 4) Control + MOSE | 2.47 | 2.46 | 4.93 |

With the support of these results, it is noted once again that the emulsifiers according to the invention appreciably increase the absorption of Tol and Tac, in vitamin E equivalent, that is by a factor of 1.92 in the case of SML, and by a factor of 1.81 in the case of SMP, as compared with the control assay 1) without emulsifier.

It is also observed that the ethoxylated emulsifier (MOSE) slightly increased the absorption of vitamin E as compared with assay 1).

### EXAMPLE 4

### Influence of an Emulsifier According to the Invention on the Release of Tac from its Support

The present example tests the effect of an emulsifier according to the invention, i.e. SMO (sorbitan monooleate), on the release of Tac which is attached to silica. To this end, use is made of Tac on a silica support, in a ratio by weight of 1:1, for the assay without emulsifier, and of Tac+emulsifier (SMO) on silica in a ratio by weight of 50:5:45.

10 g of Tac (where appropriate +SMO) on the above silica (that is a final concentration of Tac of 10 mM) are incubated for 2.5 hours, while stirring and at 38° C., under different pH conditions in the following solutions:

either 35 mM phosphate, pH 6.5, 0.15 mM NaCl

or 35 mM HCl/glycine, pH 2.5

The results compiled in Table 3 below are obtained:

TABLE 3

| | Tac without emulsifier | Tac + SMO |
|---|---|---|
| % of vitamin E | 46.5% | 54.9% |
| released at pH 2.5 | | |
| % of vitamin E | 59.6% | 72% |
| released at pH 6.5 | | |

At pH 2.5, the addition of SMO increased the release of Tac from its (silica) support by a factor of 1.18, that is by more than 18%.

At pH 6.5, this increase is 1.21 (that is more than 21%).

### EXAMPLE 5

### Influence of an Emulsifier According to the Invention on the in-vitro Hydrolysis of Tac to Tol

5.1) Influence of SMO

The present example tests the effect of an emulsifier according to the invention, i.e. SMO (sorbitan monooleate), on the hydrolysis of Tac to Tol. To this end, use is made of Tac, for the assay without emulsifier, and of Tac+SMO in a ratio by weight of 10:1.

0.5 g of Tac (where appropriate +SMO), that is a final concentration of Tac of 10 mM, is incubated for 16 hours, while stirring and at 38° C., in the following solution:

35 mM phosphate, pH 6.5, 0.15 mM NaCl, pancreatin in a ratio by weight of pancreatin:Tac of 2:1, and bile salts in a ratio by weight of bile salts:Tac of 5:1.

The pancreatin is a pancreatic extract which comprises, in particular, cholesterol ester hydrolase, and the bile salts comprise CEH activators.

The degree of release of vitamin E, as compared with the initial quantity of Tac, is measured at different incubation times under the abovementioned conditions.

The curves shown in FIG. 4 depict the degree of hydrolysis expressed as the concentration, in % (w/w), of Tol in the solution in dependence on the incubation time.

It is noted that SMO (▪) increased the degree of hydrolysis of Tac to Tol by 22% (calculated from the ratio of the slopes of the hydrolysis curves) as compared with the assay without emulsifier (♦).

5.2) Influence of SMO in Dependence on the Concentration of Bile Acids in the Incubation Solution

The present assay tests the effect of an emulsifier according to the invention, i.e. SMO (sorbitan monooleate), on the hydrolysis of Tac to Tol. For this purpose, use is made of Tac, for the assay without emulsifier, and of Tac+SMO in a ratio by weight of 10:1.

4 mg of Tac (where appropriate +SMO), that is a final concentration of Tac of 8.45 μM, are incubated for 3 hours, while stirring and at 37° C., in the following solution:

35 mM phosphate, pH 6.5, 0.15 mM NaCl, 2 mg of pancreatin, that is a ratio by weight of pancreatin:Tac of 1:2, and bile salts at varying concentrations.

The following assays are carried out:

1) Tac without emulsifier, with 10 mM bile salts

2) Tac+SMO, with 10 mM bile salts

3) Tac without emulsifier, with 20 mM bile salts

4) Tac+SMO, with 20 mM bile salts

5) Tac without emulsifier, with 50 mM bile salts

6) Tac+SMO, with 50 mM bile salts

Before incubating, and after 1 hour and 3 hours, respectively, of incubation under the abovementioned conditions, the degree of release of vitamin E, as compared with the initial quantity of Tac, is measured.

The graph in FIG. 4 depicts, for each assay, the degree of hydrolysis, expressed in the concentration, in % (w/w), of Tol in the solution.

It is noted once again that SMO strongly increases the degree of hydrolysis of Tac to Tol by activating CEH. This increase depends on the concentration of bile salts: thus, it is doubled when the concentration of bile salts is 50 mM, multiplied by 4.1 when the concentration is 20 mM, and multiplied by 4.7 when the concentration is 10 mM.

### EXAMPLE 6

### Influence of an Emulsifier According to the Invention on the Bioavailability of Vitamin E from Tac, as Measured by the Combined Influence of the Emulsifier on the Release of Tac from its Support and of the Hydrolysis of the Released Tac to Tol

The present example tests the effect of an emulsifier according to the invention, i.e. SMO (sorbitan monooleate). For this purpose, use is made of Tac on a silica support, in a ratio by weight of 1:1, for the assay without emulsifier, and of Tac+emulsifier (SMO) on silica, in a ratio by weight of 50:5:45.

8 mg of Tac (where appropriate +SMO) on the above silica (that is a final concentration of Tac of 16.9 μM) are incubated for 3 hours, while stirring and at 37° C., in the following solutions:

35 mM phosphate, pH 6.5, 0.15 mM NaCl, pancreatin (2 mg) and bile salts (20 mM).

Before incubating, and after 1 hour and 3 hours, respectively, of incubation under the aforementioned conditions, the concentration of vitamin E in the solution is measured in comparison with the initial quantity of Tac.

The results compiled in Table 4 below are obtained:

TABLE 4

| Time (minutes) | Tac without emulsifier | Tac + SMO |
|---|---|---|
| 0 | 1.21 | 6.27 |
| 60 | 1.30 | 11.56 |
| 180 | 5.25 | 9.84 |

The addition of SMO improves the bioavailability of the Tac, as assessed by the efficacy of the release of the Tac from its silica support and by the efficacy of the hydrolysis of Tac to Tol. This increase reaches more than 90% after 3 hours of incubation, resulting from an enhanced release and an accelerated hydrolysis.

### EXAMPLE 7

### Influence of the Emulsifiers on the Excretion of Vitamin E in Caecectomized Cocks

Two series of tests were carried out on caecectomized cocks. The first series comprised 32 cocks while the second comprised 51 cocks. They were fed, by “wet gavage”, with a feed which incorporated Tac (vitamin E acetate) or Tac combined, in different proportions, with one or two alimentary emulsifiers according to the invention. The Tac or the combined Tac was formulated in gelatin capsules, which were obtained by using a pipette to deposit a predetermined quantity of Tac or combined Tac in a capsule half shell and then closing the capsule with the other half shell.

48 hours after the ingestion of feed from a bowl, the faeces are collected and the excretion of the vitamin in this material is determined. To this end, the vitamin E is extracted with a vitamin E solvent, for example hexane, and the quantity extracted is then determined by HPLC chromatography. The proportion of vitamin E which has been digested is deduced from the proportion which has been excreted.

7.1) First Series

TABLE 5

| | | | | |
|---|---|---|---|---|
| Treatment | 1 | 2 | 3 | 4 |
| No. of cocks | 8 | 9 | 9 | 6 |
| Tac | 40 mg | 40 mg | 40 mg | 40 mg |
| Emulsifier | — | MLS | SMO | SMO |
| Emulsifying dose | — | 40 mg | 20 mg | 4 mg |
| Mean digestibility (%) | 24.8 | 32.5 | 29.2 | 32 |
| Standard error | 1.77 | 2.20 | 2.23 | 2.94 |
| Delta (%) | | 31.0 | 17.9 | 29.0 |

It is observed that the method according to the invention can increase the digestibility of the vitamin E by up to 31% as compared with the control.

7.2) Second Series

TABLE 6

| | Treatment | | | | | |
|---|---|---|---|---|---|---|
| | 1 | 2 | 3 | 4 | 5 | 6 |
| No. of cocks | 9 | 9 | 9 | 9 | 9 | 6 |
| Tac | 40 mg | 40 mg | 40 mg | 40 mg | 40 mg | 40 mg |
| Emulsifier | — | SML + SMO | SML + SMO | SML | SML | SML |
| Emulsifying dose | — | 20 mg | 10 mg | 20 mg | 10 mg | 20 mg |
| Mean digestibility (%) | 26.1 | 35.5 | 32.8 | 30.5 | 29 | 36.6 |
| Standard error | 1.80 | 2.63 | 1.60 | 2.73 | 1.50 | 1.55 |
| Delta (%) | | 36.6 | 25.71 | 17.1 | 11.3 | 40.5 |

As in the first series, the digestibility of the vitamin E is seen to increase when the vitamin E is administered in accordance with the invention. This increase can reach 40.5% of the digestibility obtained with Tac on its own.

### EXAMPLE 8

### Influence of the Emulsifiers on the Hydrolysis of Vitamin E Derivatives

8.1)

The conditions under which the tests are carried out are as follows:

Cell model: CaCo 2 cells

Area of the wells: 6.5 cm²

Quantity of Tac: 67 nmol/cm²

Solution employed: M40 micelle in accordance with Mathias et al. (Mathias, P. M., Harries, J. T., Muller, D. P. R. (1981): Optimization and validation of assays to estimate pancreatic ester activity using well-characterized micellar solutions of cholesteryl oleate and tocopheryl acetate. Journal of Lipid Research 22, 177-184), associated with 1.34 nmol of cholesterol ester hydrolase (EC 3.1.1.13)/cm².

Treatments:
A: Without emulsifier
B: With emulsifier (SML+SMS mixture; ratio 1/1) Tac/emulsifier ratio 100/1
C: With emulsifier (SMP+SMO mixture; ratio 1/1) Tac/emulsifier ratio 100/1

Period of incubation: 1 hour at 37° C.

Analyses: Analysis of Tac in the medium for calculating the degree of hydrolysis.

The results are presented in Table 7 below:

TABLE 7

| | A | | |
|---|---|---|---|
| | Without | B | C |
| Treatment | emulsifier | SML + MMS | SMP + SMO |
| Tac hydrolysis (%) | 10.7 | 20.9 | 21.1 |

According to this table, the presence of the emulsifiers doubled the hydrolysis of the Tac. The emulsifiers according to the invention improved the hydrolysis conditions for the cholesterol ester hydrolase.

8.2)

The conditions under which the tests are carried out are as follows:
Cell model: CaCo-2 cells
Area of the wells: 4.2 cm²
Quantity of Tac: 67 nmol/cm²
Solution employed: M40 micelle in accordance with Mathias et al.

Treatments:
A: Without emulsifier
B: With emulsifier (SMP+SMO mixture; ratio 1/1), Tac/emulsifier ratio 100/1

Period of incubation: 2 hours at 37° C.

Analyses: Analysis of Tac in the medium above the cells for the purpose of calculating the degree of hydrolysis.

The results are presented in Table 8 below:

TABLE 8

| | A | |
|---|---|---|
| | Without | B |
| Treatment | emulsifier | SML + MMS |
| Tac hydrolysis (%) | 9.7 | 14 |

The presence of the emulsifiers improved the hydrolysis of the Tac by 44%. As in 8.1), the emulsifiers improved the hydrolysis conditions for the cholesterol ester hydrolase.

### EXAMPLE 9

### Effect of an Emulsifier of the Invention on Plasma Levels of Piglets when Infused Intraduodenally

The effect of an emulsifier of the invention (50% dl-α-tocopheryl acetate+45% silica+5% monoleate sorbitane) upon the level of vitamin E in the plasma of piglets was tested. In order to exclude gastric influences or impacts of the carrier, the emulsifier of the invention was intraduodenally infused in liquid form (oil).

Three weaning piglets (initial BW 12 kg) were surgically fitted with a silicon catheter in the jugular vein as well as with a silicon cannula in the proximal duodenum according to the procedure described by Thaela et al. (1995).

A standard weaner diet without vitamin E supplementation was fed to the piglets. The animals were fed a daily feed allowance corresponding to 2.0×W^{0.75}, distributed twice in equal meals (08.00 h and 16.00). The animals were adapted for 7d to the basal diet. On D8, with the morning meal, vitamin E in the form of standard vitamin E oil (dl-α-tocopheryl acetate) or as the oil of an emulsifier of the invention (50% dl-α-tocopheryl acetate+45% silica+5% monoleate sorbitane) was administered directly according to the following experimental design.
2 treatments×3 piglets×2 repetitions (cross-over)

Treatments

R1: 800 IU Vitamin E standard oil, infused as a single bolus intraduodenally

R2: 800 IU emulsifier (50% dl-α-tocopheryl acetate+45% silica+5% monoleate sorbitane) oil, infused as a single bolus intraduodenally

Blood samples were taken 4, 8, 12, 16, 20 and 24 h postprandially, plasma was obtained and samples were pooled. After the experiment, the animals were newly adapted to the basal diet for 7d before the 2nd repetition was done accordingly. The vitamin E content in the plasma was determined by HPLC according to the method by Castan et al. (2005). Data were analysed using the ANOVA procedure of Statview.

Results and Discussion

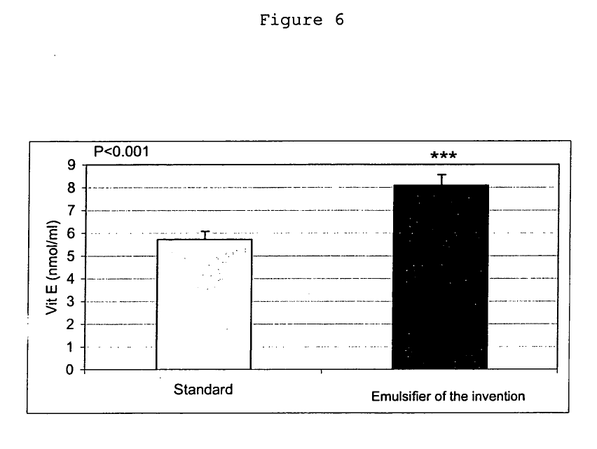

FIG. 6 represents the plasma vitamin E levels after id infusion of a single bolus of 800 IU as standard vitamin E or as the emulsifier of the invention (50% dl-α-tocopheryl acetate+45% silica+5% monoleate sorbitane).

As presented in FIG. 6, the intraduodenal (id) administration of 800 IU standard vitamin. E led to an overall plasma level of 5.72 nmol/ml (±0.36) whereas after the id administration of 800 IU of the emulsifier of the invention (50% dl-α-tocopheryl acetate+45% silica+5% monoleate sorbitane) a plasma vitamin E level of 8.08 nmol/ml (±0.47) was observed.

The low variability in the test (CV 6.2 and 5.8 for standard vitamin E and the emulsifier of the invention (50% dl-α-tocopheryl acetate+45% silica+5% monoleate sorbitane), respectively) reflect a good repeatability and therefore a valid test. It can be observed that the pigs receiving the emulsifier treatment had a significant (P<0.001) higher plasma vitamin E level when compared to piglets receiving the Vitamin E standard treatment (5.72 nmol/ml and 8.08 nmol/ml for standard vitamin E and the emulsifier of the invention, respectively). When regarding the relative increase, it can be stated that the plasma level in pigs receiving the emulsifier of the invention (50% dl-α-tocopheryl acetate+45% silica+5% monoleate sorbitane) was 42% higher as compared to the pigs receiving the standard Vitamin E.

It can be concluded that the emulsifier of the presenet invention (50% dl-α-tocopheryl acetate+45% silica+5% monoleate sorbitane) oil was at least 40% more absorbed than standard Vitamin E.

### EXAMPLE 10

### Effect of Increasing Doses of an Emulsifier of the Invention in Feed Formulations on Plasma and Liver Vitamin E Levels of Broilers

Protocol and Diets

The effect of a dietary emulsifier of the invention (50% dl-α-tocopheryl acetate+45% silica+5% monoleate sorbitane) upon the level of vitamin E in the plasma and livers of growing broilers were determined according to the following protocol:
6 treatments×50 broilers

R1: Control

R2: Control+40 IU/kg standard Vit E (in the form of dl-α-tocopheryl acetate)

R3: Control+120 IU/kg standard Vit E

R4: Control+200 IU/kg standard Vit E

R5: Control+40 IU/kg emulsifier of the invention (50% dl-α-tocopheryl acetate+45% silica+5% monoleate sorbitane)

R6: Control+120 IU/kg emulsifier of the invention (50% dl-α-tocopheryl acetate+45% silica+5% monoleate sorbitane)

Three hundred Ross male broiler chicks were divided into 6 groups and housed in 6 pens. From d0 to d14, all animals were fed the same, vitamin E free, basal diet.

The animals received the pelleted experimental diets (R1-R6) ad libitum from d15 to d34. On d35, 15 broilers per treatment, which were closest to the mean body weight, were selected. Plasma samples were collected by intracardiac puncture. Birds were then slaughtered by pentobarbital injection. Livers were dissected, deep frozen in liquid nitrogen and immediately ground. The vitamin E levels in plasma or livers were determined by HPLC according to the method by Castan et al. (2005). Data were analysed using the ANOVA procedure of Statview.

TABLE 9
Composition and characteristics of the control
diet (calculated)

| | |
|---|---|
| | Characteristics |
| Ingredients (%) | (calculated) |

| | | | |
|---|---|---|---|
| Wheat | 18.46 | AME (MJ/kg) | 12.8 |
| Corn | 37.00 | Crude protein (%) | 21.00 |
| Soybean meal | 35.54 | Methionine (%) | 0.53 |
| Soybean Oil | 4.60 | | |
| Mineral Premix | 4.4 | | |

Results and Discussion

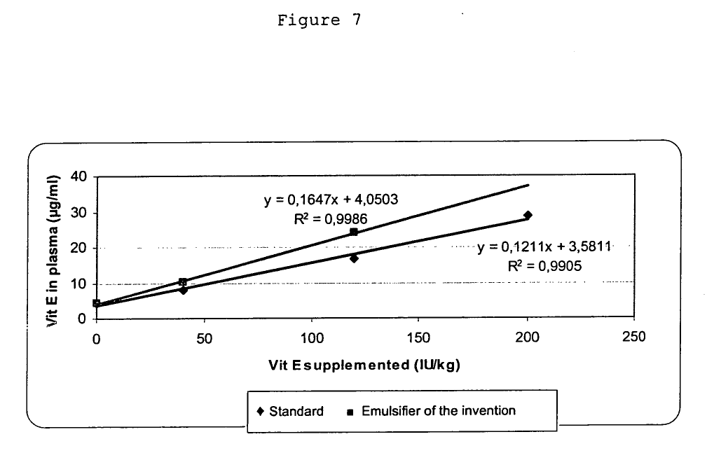

FIG. 7: Vitamin E levels in plasma of broilers fed graded levels of standard Vitamin E or emulsifier of the invention (50% dl-α-tocopheryl acetate+45% silica+5% monoleate sorbitane)

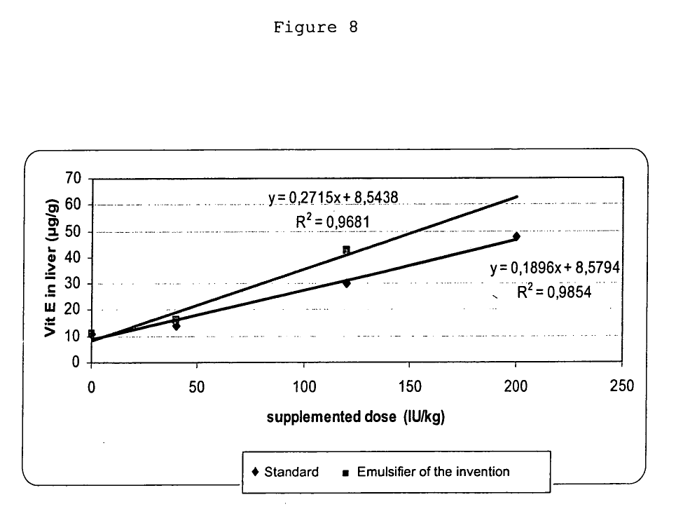

FIG. 8: Vitamin E levels in livers of broilers fed graded levels of standard Vitamin E or emulsifier of the invention (50% dl-α-tocopheryl acetate+45% silica+5% monoleate sorbitane)

The addition of standard Vitamin E to the control diet led to a linear increase of the vitamin E levels in plasma (FIG. 7) as well as in livers (FIG. 8). The addition of the emulsifier of the invention (50% dl-α-tocopheryl acetate+45% silica+5% monoleate sorbitane) also led to a linear increase of the plasma levels. For both Vitamin. E preparations, the linear increases were highly correlated, which can be seen from the very high regression coefficients (R2>0.98) for both plasma and liver levels (FIGS. 7 and 8).

The slope of the curve indicates the bioavailability of vitamin E. For plasma, the slope of the emulsifier of the invention (50% dl-α-tocopheryl acetate+45% silica+5% monoleate sorbitane) is 0.1647, where it is 0.1211 for the standard Vitamin E (FIG. 7) which means an increase in bioavailability by 38% for the plasma levels. For liver storage, the increase reaches 43% with slopes of 0.2715 and 0.1896 for the emulsifier of the invention (and standard vitamin E, respectively (FIG. 8).

From this trial it can be concluded, that an emulsifier containing 50% dl-α-tocopheryl acetate+45% silica+5% monoleate sorbitane is 40% more bioavailable than standard Vitamin E.

From both figures, it can also be calculated that to obtain the same level in plasma or liver with standard vitamin E compared to the emulsifier of the present invention, the animals need to be fed with 167 and 171 ppm of standard vitamin E compared to only 120 ppm of an emulsifier containing 50% dl-α-tocopheryl acetate+45% silica+5% monoleate sorbitane.

The emulsifier containing 50% dl-α-tocopheryl acetate+45% silica+5% monoleate sorbitane improved significantly the plasma vitamin E level (+38%) as well as the liver vitamin E levels (+43%) of broilers fed a corn-wheat based diet.

### EXAMPLE 11

### Effect of Increasing Doses of an Emulsifier of the Invention in Feed Formulations on Vitamin E Levels in Eggs

The effect of the following emulsifier: 50% dl-α-tocopheryl acetate+45% silica+5% monoleate sorbitane upon the level of Vitamin E in the egg was studied.

Protocol and Diets
4 treatments×10 layers

R1: Basal feed+10 mg/kg standard Vit E (in the form of dl-α-tocopheryl acetate)

R2: Basal feed+100 mg/kg standard Vit E

R3: Basal feed+200 mg/kg standard Vit E

R4: Basal feed+100 mg/kg emulsifier of the invention

(50% dl-α-tocopheryl acetate+45% silica+5% monoleate sorbitane)

Forty ISA brown laying hens, 23 weeks of age, were equally divided into 4 groups and individually housed in cages. The animals received the meal experimental diets (R1-R4) ad libitum for 21 days. The eggs layed on D20 to 21 were collected, weighed and stored at +4° C. The vitamin E levels in the egg yolk were determined by HPLC according to the method by Castan et al. (2005). Data were analysed using the ANOVA procedure of Statview.

TABLE 10
Composition and characteristics of the basal
diet (calculated)

| | |
|---|---|
| | Characteristics |
| Ingredients (%) | (calculated) |

| | | | |
|---|---|---|---|
| Corn | 51.50 | ME (MJ/kg) | 11.46 |
| Soybean meal | 20.30 | Crude protein (%) | 17.00 |
| Extruded soy beans | 7.50 | Methionine (%) | 0.37 |
| Wheat bran | 7.00 | Lysine | 0.91 |
| Calcium carbonate | 8.00 | Available | 0.33 |
| | | phosphorus | |
| Palm oil | 2.5 | Calcium | 3.47 |
| Mineral- & Vitamin | 3.2 | | |
| premix | | | |

Results and Discussion

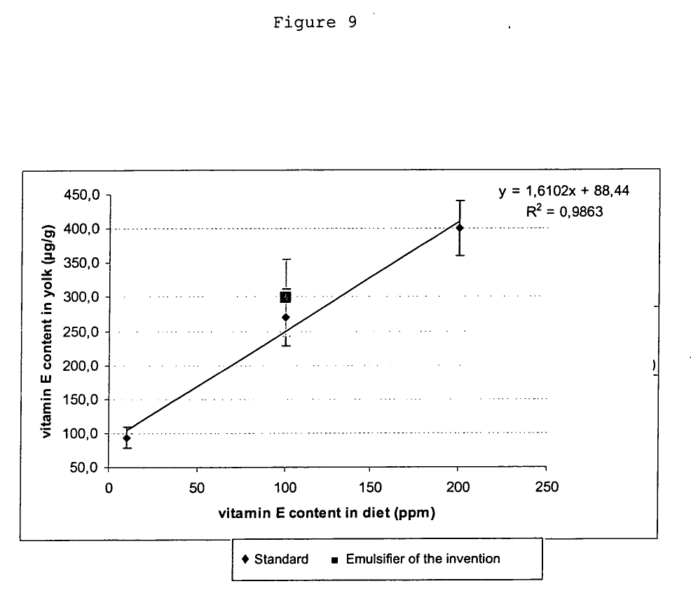

The vitamin E supplementation affected (P<0.001) the vitamin E level in eggs as well as the total content of vitamin E in the egg yolks. The increasing doses of the standard vitamin E do allow for the calculation of a regression curve of the type y=a*x+b. The value obtained for the emulsifier of the invention (50% dl-α-tocopheryl acetate+45% silica+5% monoleate sorbitane) can be inserted into this equation and therefore the equivalency of this emulsifier in relation to standard vitamin E can be calculated. As can be seen in FIG. 9, for the vitamin E level in eggs (expressed in μg/g) the regression curve for the standard vitamin E revealed the following equation: y=1.6102*x+88.44

FIG. 9: Effect of increasing doses of standard vitamin E on vitamin E levels (μg/g) in egg yolks

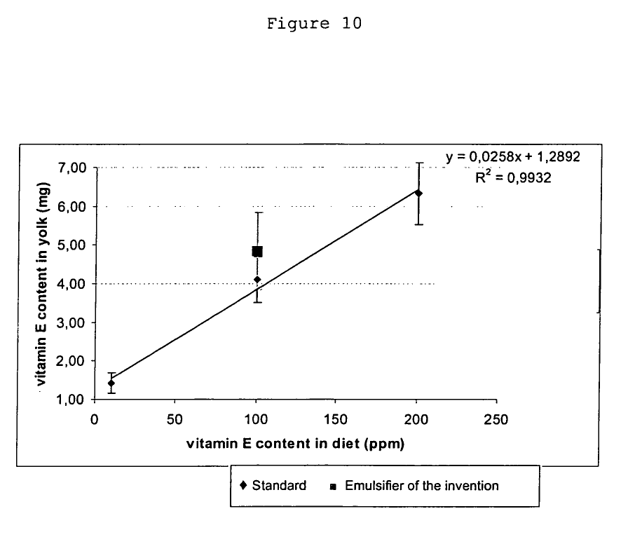

FIG. 10: Effect of increasing doses of standard vitamin E on vitamin E contents (mg) of egg yolks

For the emulsifier of interest (50% dl-α-tocopheryl acetate+45% silica+5% monoleate sorbitane), supplemented at 100 mg/kg, a vitamin E level of 297 ˜g/g (±53.1) was found. Putting this value into the equation, it can be observed that 100 mg/kg emulsifier of the invention do have the same efficacy as 130 mg/kg of standard Vit E.

For the recalculation of the total vitamin E content of the yolk, the weights of the yolks (g) have been set into relation to the vitamin E level (μg/g). The same calculation as for the vitamin E level has been done for this parameter and the following equation was obtained: y=0.0258*x+1.2892 (FIG. 10). Setting the value obtained for the emulsifier of the invention (50% dl-α-tocopheryl acetate+45% silica+5% monoleate sorbitane)(4.78 mg±0.96) into the equation, the equivalency is 138 mg/kg standard vitamin E for 100 mg/kg emulsifier of the invention. Moreover, at the supplementation level of 100 mg/kg, the emulsifier of the invention (50% dl-α-tocopheryl acetate+45% silica+5% monoleate sorbitane) led to yolks significantly (P<0.05) higher in vitamin E (4.07 mg±0.57 for standard vitamin E, 4.78 mg±0.96 for the emulsifier of the invention).

It can be concluded, that the supplementation of a corn-based layer feed with 100 mg/kg emulsifier of the invention (50% dl-α-tocopheryl acetate+45% silica+5% monoleate sorbitane) leads to the same vitamin E contents of egg yolks as the supplementation with 138 mg/kg of standard vitamin E.

The supplementation of a corn-based layer feed with 100 mg/kg emulsifier of the invention (50% dl-α-tocopheryl acetate+45% silica+5% monoleate sorbitane) leads to the same vitamin E contents of egg yolks as the supplementation with 138 mg/kg of standard vitamin E.

### EXAMPLE 12

### Effect of an Emulsifier of the Invention on Plasma Kinetics in Cockerels

Protocol and Diets

The effect of an emulsifier of the invention (50% dl-α-tocopheryl acetate+45% silica+5% monoleate sorbitane) upon the plasma kinetics of Vitamin E in cockerels was tested according to the following protocol: 2 treatments×5 cockerels

R1: 40 IU/bird standard Vit E (in the form of dl-α-tocopheryl acetate)

R2: 40 IU/bird emulsifier of the invention (50% dl-α-tocopheryl acetate+45% silica+5% monoleate sorbitane)

Ten adult cockerels were housed in individual metabolic cages and distributed into 2 groups. Prior to the experiment, birds were fasted for 24 h. For the experiment, birds were force-fed a gelatine capsule containing the vitamin E preparations (R1 and R2) in the form of Vitamin E oil. Plasma samples were obtained from the wing vein of the animals according to the sampling scheme presented in FIG. 1. The Vitamin E levels in plasma were determined by HPLC according to the method by Castan et al. (2005). Data were analysed using the ANOVA.

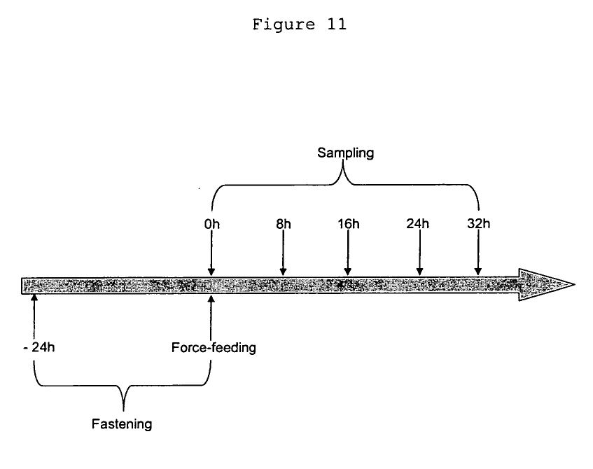

FIG. 11: Sampling scheme for obtaining plasma samples

Results and Discussion

The basal level of Vitamin E was determined in order to be able to do the calculation of increase via differentiation. The basal level was found to be at 8.2 nmol/ml.

When calculating the amount of absorbed Vitamin E by calculating the area under the curve, it can be seen that after 8 h the animals receiving the following emulsifier: 50% dl-α-tocopheryl acetate+45% silica+5% monoleate sorbitane, had 121% more Vit E uptaken in comparison to the animals given the standard Vitamin E oil. After 16 h there was still 69% more Vitamin E uptake; after 24 h a plus of 41% was found.

These findings demonstrate a greater absorption of Vitamin E when given in the form of the emulsifier of the present invention. The differences between the treatments are the most pronounced at the beginning of the trial; after 24 h they become smaller due to the fact that the animals have not been fed and are in a status of fasting.

It can be concluded from this study that Vitamin E oil in the form of an emulsifier of the present invention is at minimum 40% more bioavailable as standard Vitamin E oil.

### EXAMPLE 13

### Effect of a Dietary Emulsifier of the Invention on Vitamin E Levels in Liver and Muscle of Piglets

The effect of an emulsifier of the invention (50% dl-α-tocopheryl acetate+45% silica+5% monoleate sorbitane) upon the level of vitamin E in the livers as well in the muscle (ham) was tested according the following protocol: 3 treatments×10 piglets

R1: Basal diet+20 IU/kg standard Vit E (in the form of dl-α-tocopheryl acetate)

R2: Control+150 IU/kg standard Vit E

R3: Control+150 IU/kg emulsifier of the invention (50% dl-α-tocopheryl acetate+45% silica+5% monoleate sorbitane)

Thirty crossbred weanling piglets (8.0 kg live weight) were divided into three groups (R1, R2, R3), kept individually and distributed according to a completely randomized block design. The piglets were fed ad libitum for 14 days on a corn-wheat based diet (Table 11). The diet was supplemented with either 20 IU/kg (R1) or 150 IU/kg (R2) standard DL-α-tocopheryl acetate. R3 piglets were fed a diet supplemented with 150 IU/kg of emulsifier containing 50% dl-α-tocopheryl acetate+45% silica+5% monoleate sorbitane. On day 15 of the experiment the animals were slaughtered and their liver dissected, immediately frozen under liquid nitrogen and ground to powder. Muscle samples (muscularis glutaeus maximus) of about 9 cm2 were obtained from the left hind leg and frozen immediately. The vitamin E content of the samples was determined by HPLC (Castan et al. 2005) and data were statistically analysed using one-way ANOVA.

TABLE 11
Composition and characteristics of the
control diet (calculated)

| | |
|---|---|
| | Characteristics |
| Ingredients (%) | (calculated) |

| | | | |
|---|---|---|---|
| Wheat | 15.00 | DE (kcal/kg) | 3400 |
| Corn | 35.00 | Lys (%) | 1.5 |
| Soybean meal | 25.00 | | |
| Skimmed milk | 15.0 | | |
| powder | | | |
| Sugar | 5.0 | | |
| Mineral Premix | 5.0 | | |

Results and Discussion

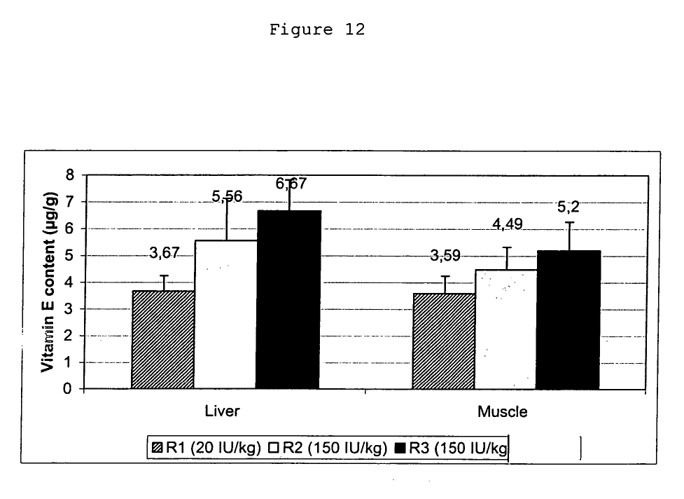

Adding an emulsifier of the invention (50% dl-α-tocopheryl acetate+45% silica+5% monoleate sorbitane) to the diet increased vitamin E content in liver by 81% (P<0.02) and in muscle by 44% (P<0.01) as compared to contents in piglets fed 20 IU/kg DL-α-tocopheryl acetate. Vitamin E contents increased in liver (51%) and muscle (35%) when DL-α-tocopheryl acetate was increased from 20 IU/kg to 150 IU/kg in the diet (P<0.05). The relative comparison of the slopes of the regression curves enables an evaluation of the bioavailability of vitamin E from the two sources. Therefore, a regression curve has been made for both vitamin E sources using the point at 20 IU/kg and the corresponding point at 150 IU/kg (R1 and R2 for standard DL-α-tocopheryl acetate and for R1 and R3 for 50% dl-α-tocopheryl acetate+45% silica+5% monoleate sorbitane). The division of the slopes of the curves gives an evaluation of the difference in bioavailability. The comparison of the slopes of the curves for vitamin E contents in livers (1.89 for standard DL-α-tocopheryl acetate and 3.00 for the emulsifier of the invention) suggest an increase in relative bioavailability for the emulsifier of the invention of 37%. For muscle samples, the slopes of the curves were 0.89 for standard DL-α-tocopheryl acetate and 1.61 for the emulsifier of the invention. As a result, an increase in relative bioavailability of the emulsifier of the invention of 44% was estimated (FIG. 12).

FIG. 12: Vitamin E levels in plasma of broilers fed graded levels of standard Vitamin E or an emulsifier of the invention

From this trial it can be concluded, that an emulsifier of the invention (50% dl-α-tocopheryl acetate+45% silica+5% monoleate sorbitane) is 40% more bioavailable than standard Vitamin E.

1. Non-therapeutic animal husbandry method, consisting in administering, by the oral route to reared mono-gastric animals, a stable derivative of vitamin E either on its own or mixed with an additive and/or a feedstuff, with the said derivative being hydrolyzable into the assimilable form of vitamin E, wherein the said vitamin E derivative is administered to the animals in the presence of at least one alimentary emulsifier selected from the non-ethoxylated esters of sorbitol and fatty acids.

2. Method according to claim 1, wherein the derivative of vitamin E and the said at least one emulsifier are administered to the animals concomitantly.

3. Method according to claim 1, wherein the said emulsifier is administered to the animals before the vitamin E derivative has been administered to the animals.

4. Method according to claim 2, wherein the derivative of vitamin E and/or the said at least one emulsifier are mixed with the feedstuff.

5. Method according to claim 1, wherein the said at least one emulsifier is selected from the esters of sorbitol and fatty acids which have a saturated or unsaturated hydrocarbon chain of at least 11 carbon atoms.

6. Non therapeutic alimentary formulation for animal nutrition comprising a stable derivative of vitamin E, with the said derivative being hydrolyzable into the assimilable form of vitamin E, further comprising at least one alimentary emulsifier selected from the non-ethoxylated esters of sorbitol.

7. Formulation according to claim 6, further comprising a silica support.

8. Formulation according to claim 6, further comprising two alimentary emulsifiers selected from the non-ethoxylated esters of sorbitol and fatty acids.

9. Formulation according to claim 6, wherein the ratio by weight of the emulsifier, or of the emulsifiers, to the vitamin derivative varies from 10/1 to 1/200.

10. Formulation according to claim 9, wherein the ratio by weight of the emulsifier, or of the emulsifiers, to the vitamin E derivative is between 1/5 and 1/100.

11. Formulation according to claim 6, wherein the said at least one emulsifier is selected from the esters of sorbitol and fatty acids which have a saturated or unsaturated hydrocarbon chain of at least 11 carbon atoms.

12. Formulation according to claim 6, wherein the derivative of vitamin E is a vitamin E ester.

13. Formulation according to claim 12, wherein the derivative of vitamin E is vitamin E acetate.

14. Formulation according to claim 6, wherein the said at least one emulsifier is a sorbitol ester selected from the monolaurate, the monopalmitate, the monostearate, the monooleate and the tristearate.

15. Formulation according to claim 6, further comprising vitamin E acetate and at least one emulsifier selected from sorbitol monolaurate and sorbitol monooleate.

16. Formulation according to claim 6, further comprising vitamin E acetate and sorbitol monooleate on silica in a ratio by weight of 50:5:45.

17. (canceled)
